(19) United States
(12) Patent Application Publication
(10) Pub. No.: US 2005/0244343 A1
(43) Pub. Date: Nov. 3, 2005

(54) Oral care products comprising silica

(76) Inventors: Michael C. Withiam, Landenberg, PA (US); Dev K. Mehra, Furlong, PA (US); John M. Cornelius, Forest Hill, MD (US)

Correspondence Address: Carlos Nieves, Esq.;J. M. Huber Corporation, 333 Thornall Street, Edison, NJ, 08837-2220, US

(21) Appl. No.: 10/835,733

(22) Filed: Apr. 30, 2004

(51) Int. Cl. A61K 7/16; A61K 9/20; A61K 9/46

(52) U.S. Cl. 424/49; 424/464

(57) ABSTRACT

A rapidly disintegrating oral care tablet is provided. The tablet comprises: a silica; a super disintegrant; and a sugar alcohol. When immersed in water the tablet has a friability of less than about 2% and disintegrates in less than about 60 seconds.

### BACKGROUND OF THE INVENTION

Many consumer products, such as health and personal care products, are manufactured and packaged in solid, compacted form. The solid, compacted product form has several advantages over other product forms, such as relative ease of manufacture and durability in packaging and shipment and convenience in use and in storing for retailers and consumers alike. The compressed tablet form is particularly well-suited for oral care and hygiene.

However, in certain situations it would be beneficial if the tablet would disintegrate in the mouth so that tooth cleaning could be affected without the necessity of having access to a toothbrush or to water. For example, hikers, campers, boaters, or people traveling or eating in public places, could use an oral care tablet that rapidly disintegrates in the mouth providing a convenient and effective solid form delivery system for tooth cleaning and mouth freshening.

Unfortunately, most tablets do not readily disintegrate in the mouth, but instead disintegrate in a slow and uneven fashion, for example when chewed. Given the forgoing there is a continuing need for solid form oral care preparations that rapidly disintegrate in the mouth and that are not friable under packaging and shipping conditions.

### BRIEF SUMMARY OF THE INVENTION

The present invention includes a rapidly disintegrating oral care tablet comprising (a) about 10% to about 80% silica, (b) about 20% to about 80% of a sugar alcohol and (c) about 1% to about 30% of a super-disintegrant.

### DETAILED DESCRIPTION OF THE INVENTION

All parts, percentages and ratios used herein are expressed by weight unless otherwise specified.

All publications, patent applications and issued patents mentioned herein are hereby incorporated in their entirety by reference.

The present invention relates to personal care products that are oral care products in solid or semi-solid form such as dentifrices, toothpastes, and breath-fresheners; these personal care products include silica.

The oral care products of the present invention typically contain from about 10% to about 80% silica, preferably from about 15% to about 50%, about 20% to 80% sugar alcohol, preferably about 20% to about 70%, and about 1% to about 30% of a super disintegrant, preferably about 3% to about 15%, more preferably about 3% to 5%.

The silica component of the present silica substrate is preferably an amorphous precipitated silica. Preferred precipitated silicas include the following products available from the J. M. Huber Corporation, Edison, N.J.: Zeo® 49, Zeofree® 153, Zeothix® 265, Zeothix 95, and Zeothix 177, Zeodent® 103, Zeodent® 113, Zeodent® 114, Zeodent® 115, Zeodent® 118, Zeodent® 119, Zeodent® 165, and Zeodent® 9175. Silicas suitable for use in the present invention, as well as processes suitable for preparing them, are set forth in U.S. Pat. Nos. 3,893,340, 4,340,583, 5,225,177, and 6,616,916, as well as U.S. patent Publication No. 2003/0131536 A1. Additionally, precipitated amorphous silicas are available from Ineos Silicas, Warrington, England, marketed under Sorbosil; from Rhodia Silica Systems, Lyon, France, marketed under Tixosil® and Oralsil®V and from Degussa AG, Germany marketed under Sident.

In addition to precipitated silica, the silica may also be selected from (without intending to be limiting) amorphous silicas such as silica gel, and pyrogenic silica. Suitable pyrogenic silicas include Aerosil® products available from the Degussa AG, Germany; and Cab-O-Sil® products available from Cabot Corporation, Bellerica, Mass. Suitable silica gels include Silcron® products available from Millennium Inorganic Chemicals Corporation, Baltimore Md.; and Syloid®, Sylodent®R, Syloblanc® and Sylox® products available from Grace & Co., Davison Chemical Division, Baltimore, Md.

The sugar alcohol provides multiple functions to the rapidly disintegrating oral care tablet. The sugar alcohol provides good aesthetic properties to the dissolved oral care tablet such as taste and “mouth texture” or body; aids in rapid tablet disintegration; and serves as a tablet filler. Suitable sugar alcohols include glycerin (glycerol), erythritol, xylitol, sorbitol, maltitol, mannitol, lactitol, and the like, used singly and in combinations, with mannitol and sorbitol preferred.

The super disintegrant facilitates the break-up of a tablet when it is placed in an aqueous environment, such as the mouth. Super disintegrants in contact with water swell, wick-in water or otherwise provide a disruptive force to a tablet causing it to break apart. Suitable super disintegrants include one or more of sodium starch glycolate, available as e.g. Explotab and Explosol; croscarmellose sodium (cross-linked sodium carboxymethyl cellulose) available as e.g. Ac-Di-Sol® and Nymcel® ZSX; and cross-linked polyvinylpyrolidones available as e.g. Polyplasdone XL.

In addition to the aforementioned ingredients, the oral care products of the present invention may also include several other ingredients such as additional disintegration aids, organoleptic enhancers, additional abrasives, thickening agents, (also sometimes known as thickeners, binders, gums, or stabilizing agents), therapeutic agents, and preservatives.

These solid formed oral care preparations may also include one or more disintegration aids, in addition to the super disintegrant. Suitable disintegration aids include natural, modified or pregelatinized starch; natural or chemically-modified cellulose; microcrystalline cellulose; gum, especially agar gum, and guar gum; alginic acid or salts thereof; acetates and citrates; sugars (especially sucrose, amylose, dextrose and lactose); aluminum oxide; synthetic polymers such as methacrylic acid-divinylbenzene copolymer, as well as effervescent disintegrating systems. Typical levels of disintegration aids in the inventive oral care preparations are from about 0.5% to about 15% of the formulation, preferably from about 1% to about 5%.

The inventive oral care compositions may also contain one or more organoleptic enhancing agents. Organoleptic enhancing agents include humectants, sweeteners, surfactants, flavorants, colorants and effervescing agents.

Humectants serve to add body or “mouth texture” to a dentifrice. In addition to the previously mentioned sugar alcohols, suitable humectants include glycerin, polyethylene glycol (at a variety of different molecular weights), propylene glycol, and hydrogenated starch hydrolyzates, as well as mixtures of these compounds.

Sweeteners may be added to the dentifrice composition to impart a pleasing taste to the product. Suitable sweeteners include saccharin (as sodium, potassium or calcium saccharin), cyclamate (as a sodium, potassium or calcium salt), aspartame, acesulfane-K, thaumatin, neohisperidin dihydrochalcone, ammoniated glycyrrhizin, dextrose, maltodextrin, sucralose, fructose, levulose, sucrose, mannose, and glucose. Typical levels of sweeteners are from about 0% to about 5% of a dentifrice composition.

Surfactants are used in the compositions of the present invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate,, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents consist chemically of mixtures of aldehydes, ketones, esters, phenols, acids, and aliphatic, aromatic and other alcohols.

Colorants may be added to improve the aesthetic appearance of the product. Suitable colorants are selected from colorants approved by appropriate regulatory bodies such as the FDA and those listed in the European Food and Pharmaceutical Directives and include pigments, such as TiO₂, and colors such as FD&C and D&C dyes.

The oral care product may also contain an effervescent agent to provide aesthetic properties to the tablet. Preferably effervescence is provided by reaction of a carbonate salt such as calcium carbonate, sodium carbonate, sodium bicarbonate, potassium carbonate or potassium bicarbonate with an acid such as citric acid, tartaric acid or malic acid.

In addition to silica, the oral care tablet may contain additional abrasives. Suitable abrasives include precipitated and ground calcium carbonate, calcium metasilicate, calcium pyrophosphate, dicalcium phosphate, dicalicum phosphate dihydrate, aluminum silicate, alumina, calcined alumina, bentonite, particulate thermosetting resins and other suitable abrasive materials known to a person of ordinary skill in the art. The abrasive may be used alone or in combination with other abrasives. Typical levels of abrasives in the inventive dentifrice formulation are from about 2% to about 60%, preferably from about 2% to about 10%.

Thickening agents are useful in the oral care products of the present invention to provide an aesthetically pleasing texture when the composition disintegrates in the mouth. Suitable thickening agents include silica thickeners such as J. M. Huber Corporation Zeodent® precipitated silica products and silica gels available from Davison Chemical Division of W. R. Grace Corporation, Baltimore, Md.; natural and synthetic clays such as hectorite clays; lithium magnesium silicate (laponite) and magnesium aluminum silicate (Veegum); starch; glycerite of starch; as well as mixtures of these compounds. Typical levels of thickening agents are from about 0% to about 15% of an oral care composition.

Therapeutic agents are optionally used in the compositions of the present invention to provide for the prevention and treatment of dental caries, periodontal disease and temperature sensitivity. Examples of therapeutic agents, without intending to be limiting, are fluoride sources, such as sodium fluoride, sodium monofluorophosphate, stannous fluoride, potassium fluoride, sodium fluorosilicate, ammonium fluorosilicate and the like; condensed phosphates such as tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quartemary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives may be also be optionally added to the compositions of the present invention to prevent bacterial growth. Suitable preservatives approved for use in oral compositions such as methylparaben, propylparaben and sodium benzoate may be added in safe and effective amounts.

The oral care products may additionally contain other optional ingredients typically used in tablet making such as glidants to provide even flow to the granulation to be tabletted, e.g. amorphous silica such as Zeopharm® 80 (J. M. Huber Corporation, Edison, N.J.) and Cab-O-Sil® M5 (Cabot Corporation, Billerica, Mass.); die release aids, also known as lubricants, such as magnesium stearate (available as HYQUAL® NF from Mallinckrodt, Inc., St. Louis, Mo.) to enable tablets to be released from within the tablet machine die, anti-adherents, such as stearic acid, to facilitate separation of tablets from punch faces; and fillers such as microcrystalline cellulose, such as Avicel 101 (FMC Biopolymers, Philadelphia, Pa.) and Omnicel 102 (Functional Foods, Englishtown, N.J.).

All tablet formulation ingredients, except the lubricant, are weighed together and mixed. Thereafter, the lubricant is geometrically diluted with the just prepared tablet mixture and then added back to the mixture. This step is typically necessary to homogeneously incorporate the hydrophobic lubricant into the tablet mixture.

The tablets are then manufactured by using a tableting compacting process. A standard single stroke or a rotary press may be used. The tablets prepared according to this invention may be of any geometrical shape, such as round, square, triangular, or caplet-shaped, and of any size suitable for human or animal use.

The invention will now be described in more detail with respect to the following, specific, non-limiting examples.

### Oral Care Tablet Preparation

Oral care tablets were prepared by weighing all formulation ingredients together, except the lubricant magnesium stearate, on a weighing pan. Typically, a tablet formulation was 300 g to 500 g total weight, in order to prepare multiple tablets for testing. The combined ingredients were passed through a 20 mesh (850 μm) sieve to remove any lumps and then bag blended, by gentle inversion in a plastic bag for about 30 seconds of the formulation ingredients previously weighed. The resulting mixture was transferred to a PK-V blender (twin shell dry blender model 014-215-0053, available from Patterson Kelly, East Stroudsburg, Pa.) and mixed for 10 minutes. The magnesium stearate lubricant was then geometrically diluted with the mixture and then added back to the PK blender and all ingredients mixed together for an additional 5 minutes.

Tablets were formed from the resulting formulation on a 8-station Piccola rotary tablet press available from Riva S.A., Argentina, fitted with 10 mm standard concave die punches compacting over a range of compression forces, expressed in kN. Tablet weight was set at 400 mg by adjusting the tablet press.

Excipients, used in the following examples, were obtained as follows.

| Excipient | Grade | Company |
|---|---|---|
| Precipitated Silica | Zeodent ® 113 | J. M. Huber Corp., Edison, NJ |
| | Zeodent ® 119 | |
| | Zeodent ® 165 | |
| | Zeodent ® 9175 | |
| Silica gel | Sylodent ® 756 | Grace Davison, Baltimore, MD |
| Mannitol | Pearlitol ® | Roquette Freres, Lestrem, France |
| | 200SD | |
| Compressible | Nu-Tab ® 4000 | Chr Hansen, Vineland, NJ |
| sugar | | |
| Crospovidone | Polyplasdone ® | International Specialty Products, |
| | XL-10 | Wayne, NJ |
| Sodium starch | Explotab ® | Penwest Pharmaceuticals Co., |
| glycolate (SSG) | Patterson, NY | |
| Microcrystalline | Omnicel ® 102 | Functional Foods, Englishtown, NJ |
| cellulose (MCC) | | |
| Microcrystalline | Avicel ® 101 | FMC Biopolymer, Philadelphia, |
| cellulose (MCC) | | PA |
| Sodium lauryl | | Aceto Corporation, Lake |
| sulfate | | Success, NY |
| Fumed Silica | Cab-O-Sil ® M5 | Cabot Corporation, Bellerica, MA |
| glidant | | |
| Magnesium | Hyqual ® NF | Mallinckrodt, Inc., St. Louis, MO |
| stearate | | |

### Tablet Test Methods

All tablets were prepared 24 hours before testing hardness, disintegration time and friability.

Tablet hardness (H) expressed in kP, for each formulation, was measured on 5 tablets utilizing a Erweka TBH30 instrument (Milford, Conn.) and the result reported was an average of 5 measurements.

Tablet disintegration time was determined by placing 6 tablets (each tablet in a separate tube) in an Erweka ZT72 disintegrator (Milford, Conn.). The tablets were repeatedly immersed in 37° C. deionized water at a rate of 30 strokes/min. until the tablets disintegrated, as detected and recorded by the instrument. The reported result was an average of the 6 measurements.

Tablet friability was determined by placing 10 tablets in a Distek, Inc. Friabilator DF-3 (North Brunswick, N.J.) set for 100 revolutions. The % friability is calculated from the amount of tablet weight lost (friable) by weighing the tablets before and after rotation.

### EXAMPLES 1-6

In theses examples, oral care tablet formulations were made with silica, a super disintegrant, a sugar alcohol and other ingredients typically found in oral care formulations and in tablet formulations. These formulations were prepared according to the procedure described above with the amounts of ingredients identified in Table 1.

TABLE 1
Tablet Formulations

| | Example # | | | | | |
|---|---|---|---|---|---|---|
| | 1 | 2 | 3 | 4 | 5 | 6 |
| % Zeodent 165 | 4.5 | | | | | |
| % Zeodent 113 | | 10 | | 25 | 27 | 27 |
| % Sylodent 756 | | | 10 | | | |
| % Zeodent 9175 | | | | 3 | | |
| % Pearlitol 200SD | 32.69 | 63.25 | 63.25 | 27 | 51.25 | 66.25 |
| Mannitol | | | | | | |
| % Omnicel 102 | 60.71 | 20 | 20 | 17 | 15 | 0 |
| MCC | | | | | | |
| % Explotab | 0.5 | | | | | |
| (SSG) | | | | | | |
| % Polyplasdone XL | | 5 | 5 | 10 | 5 | 5 |
| Crospovidone | | | | | | |
| % Sodium lauryl | | | | 2 | | |
| sulfate | | | | | | |
| % Flavor | | | | 6.5 | | |
| % STPP | | | | 5 | | |
| % Aspartame | | | | 3 | | |
| % Cab-O-Sil M5 | 1 | 1 | 1 | 1 | 1 | 1 |
| silica glidant | | | | | | |
| % Magnesium | 0.6 | 0.75 | 0.75 | 0.5 | 0.75 | 0.75 |
| Stearate | | | | | | |

Tablets weighing 400 mg each were prepared according to the procedure described above. Each formulation was compressed into tablets at different compression forces for each respective formulation. The tablet hardness (H), disintegration time (DT) and Friability were determined according to the procedures described above for tablets pressed at different compression forces with the results summarized in Table 2 below.

TABLE 2
Tablet Properties

| Formulation | Compression | Hardness | DT | % |
|---|---|---|---|---|
| No. | Force (kN) | (kP) | (s) | Friability |
| 1 | 2.2 | 2.87 | 8 | 0.50 |
| 1 | 5.0 | 7.85 | 15 | 0 |
| 1 | 6.6 | 10.1 | 18 | 0 |
| 2 | 5.1 | 3.06 | 10 | 0.44 |
| 3 | 3.8 | 2.73 | 7 | 0.66 |
| 3 | 7.9 | 8.13 | 10 | 0.07 |
| 3 | 9.4 | 10.13 | 11 | 0.15 |
| 4 | 8.5 | 2.12 | 8 | 2.3 |
| 4 | 17 | 5.69 | 13 | 0.44 |
| 4 | 24.1 | 9.95 | 25 | 0.25 |
| 5 | 7.70 | 3.1 | 9 | 1.11 |
| 5 | 16.7 | 7.83 | 13 | 0.40 |
| 5 | 18.7 | 10.7 | 14 | 0.30 |
| 6 | 8.3 | 2.06 | 8 | 1.06 |
| 6 | 17.4 | 6.5 | 11 | 0.63 |
| 6 | 22 | 7.56 | 11 | 2.66 |

It is seen from the data in Table 2 above that all of these tablet formulations could be compressed into non-friable tablets with disintegration times of about 10 seconds. Generally, tablets with a friability of greater than 1% either were not compressed with enough force for the tablet to remain intact or the tablets capped. Example 2 which contained 10% silica, about 63% mannitol and 20% MCC was only compressed into tablets at one compression force, since the tablet ejection force exceeded 1000N.

### COMPARATIVE EXAMPLES

For comparison, tablet formulations were prepared as described above, but each formulation was missing an essential ingredient selected from a sugar alcohol, a super disintegrant and silica. For instance, Comparative Example 1 (C1) contained silica and a super disintegrant, but compressible sugar instead of a sugar alcohol. Comparative Example 2 (C2) contained silica and the sugar alcohol mannitol, but no super disintegrant. Comparative Example 3 (C3) contained a sugar alcohol and a super disintegrant, but no silica. The formulations are summarized in Table 3 below.

TABLE 3
Comparative Example Tablet Formulation

| | Example No. | | |
|---|---|---|---|
| | C1 | C2 | C3 |
| Zeodent ® 113 Silica | 27 | 27 | 0 |
| Nu-Tab 4000 | 51.25 | 0 | 49 |
| Compressible sugar | | | |
| % Pearlitol 200SD | 0 | 56.25 | 0 |
| Mannitol, % | | | |
| % Avicel 101 MCC | 15 | 15 | 40 |
| % Explotab | 0 | 0 | 10 |
| (SSG) | | | |
| % Polyplasdone XL-10 | 5 | 0 | 0 |
| Crospovidone | | | |
| % Cab-O-Sil M5 | 1 | 1 | 0 |
| Silica glidant | | | |
| % Magnesium Stearate | 0.75 | 0.75 | 1 |

Each formulation was compressed into tablets at different compression forces for each respective formulation. Comparative Examples 1 and 2 tablets were prepared as described above. For Comparative Example 3 formulation, 500 mg tablets were made by direct compression in an Angstrom pellet press at forces of 3.6, 4.4, and 8.9 kN. The Angstrom press mould had a circular shape and a diameter of 1.4 cm.

For Comparative Examples 1 and 2, the tablet hardness, disintegration time (DT) and % Friability were determined according to the procedures described above for tablets pressed at different compression forces. Since Comparative Example 3 tablets were prepared manually, only disintegration time was determined. Comparative Example 3 tablets were immersed in deionized water at 37° C. and the time (seconds) required for initial fracture of the tablet was recorded for disintegration time. Results for all three comparative examples are summarized in Table 4 below.

TABLE 4
Tablet Properties

| Formulation | Compression | Hardness | | % | |
|---|---|---|---|---|---|
| No. | Force (kN) | (kP) | DT (s) | Friability | Comments |
| C1 | 10.6 | 2.32 | 6 | 3.675 | Tablets |
| C1 | 16.1 | 3.24 | 6 | 10.138 | Capped |
| C2 | 8.4 | 3.36 | 52 | 0.601 | Poor flow |
| C2 | 15 | 7.05 | 139 | 0.442 | |
| C2 | 20 | 9.97 | 227 | 0.391 | |
| C3 | 3.6 | Not tested | 4 | Not tested | Capped |
| C3 | 4.4 | Not tested | >300 | Not tested | |
| C3 | 8.6 | Not tested | >300 | Not tested | |

It is seen from the above data that tablets without silica (C3) and without a super disintegrant (C2) had longer disintegration times than tablets of comparable hardness made according to the present invention. The tablets made with compressible sugar instead of the sugar alcohol all capped yielding very high friability.

### COMPARATIVE EXAMPLE 4

Oral care tablets were formed as in Example II of WO 99/33437 without the non-essential ingredients: color, sodium fluoride, cetyl pyridinium chloride and flavor. Since this patent application was silent as to the compression forces used to form the tablets, several different compression forces were used for tablet formation. Since this formulation would not flow evenly to feed an automatic tableting press, tablets were formed by manually pressing the resulting mixture in an Angstrom press fitted with a 13-mm diameter die at 2000 psi, 1000 psi and 500 psi. The formulation ingredient amounts are given below in Table 5. Example II of WO99/33437 is reproduced herein as Comparative Example 4 (C4).

### Table 5

Tablet Formulation

| | Comparative |
|---|---|
| | Ex. 4 |
| Sorbitol, g | 10 |
| Mannitol, g | 46.7 |
| Precipitated Silica, g | 30 |
| Zeodent ® 119 | |
| Sodium lauryl sulfate, g | 1 |
| Potassium citrate, g | 1 |
| Sodium saccharine, g | 0.13 |
| Xanthan gum, g | 0.1 |
| Sodium CMC, g | 0.15 |
| Synthetic silicate, g | 4.6 |
| Zeodent ® 165 | |
| Magnesium stearate, g | 2.5 |
| Talc, g | 2 |

Disintegration time was determined on Comparative Example 4, compressed at 500 psi. Disintegration time was determined by placing 3 tablets in separate tubes in an Erweka ZT72 disintegrator. The tablets were repeatedly immersed in 37° C. deionized water at a rate of 30 strokes per minute until the tablets disintegrated, as detected and recorded by the instrument. These C4 tablets had a hardness of 2.2 kP and a disintegration time of over 9 minutes.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A rapidly disintegrating oral care tablet comprising:
a silica;
a super disintegrant; and
a sugar alcohol;
wherein the tablet has a friability of less than about 2% and disintegrates when immersed in water in less than about 60 seconds.

2. The oral care tablet according to claim 1, wherein the tablet comprises about 10% to about 80 wt % of silica.

3. The oral care tablet according to claim 1, wherein the super disintegrant is selected from one or more of sodium starch glycolate, croscarmellose sodium, and crospovidone.

4. The oral care tablet according to claim 1, wherein the tablet comprises about 1 wt % to about 30 wt % of the super disintegrant.

5. The oral care tablet according to claim 1, wherein the tablet comprises about 1 wt % to about 3 wt % of the super disintegrant

6. The oral care tablet according to claim 1, wherein the sugar alcohol is selected from one or more of sorbitol, mannitol, xylitol, erythritol, maltitol, and lactitol.

7. The oral care tablet according to claim 1, wherein the tablet comprises about 20 wt % to about 80 wt % of the sugar alcohol.

8. The oral care tablet according to claim 1, wherein the tablet friability is less than 1%.

9. The oral care tablet according to claim 1, wherein the tablet, when added to water at 37° C. disintegrates in less 40 seconds.

10. The oral care tablet according to claim 1, wherein the tablet, when added to water at 37° C. disintegrates in less 20 seconds

11. The oral care composition according to claim 1, further comprises one or more ingredients selected from the group consisting of: organoleptic enhancing agents, abrasives, disintegration aids, preservatives, therapeutic agents and thickening agents.

12. The oral care composition according to claim 11, wherein the organoleptic enhancing agent comprises one or more ingredients selected from the group consisting of humectants, sweeteners, flavorants, surfactants, colorants and effervescent agents.

13. A rapidly disintegrating oral care tablet comprising:
about 10 wt % to about 80 wt % silica;
about 1 wt % to about 15 wt % super disintegrant;
about 20 wt % to about 80 wt % sugar alcohol; and
about 0.1 wt % to about 5 wt % surfactant; wherein the rapidly disintegrating oral care tablet has a friability of less than about 2% and the tablet disintegrates when immersed in water in less than about 60 seconds.

14. The rapidly disintegrating oral care tablet according to claim 13, further comprising a flavorant.
